(19) United States
(12) Patent Application Publication
(10) Pub. No.: US 2009/0285903 A1
(43) Pub. Date: Nov. 19, 2009

(54) METHODS FOR DENDRITIC CELL THERAPY USING PHARMACOLOGICALLY ACTIVE MICROCARRIERS

(75) Inventor: Steven F. Josephs, Escondido, CA (US)

Correspondence Address: Coastal Patent, LLC, P.O.BOX 232340, San Diego, CA, 92193, US

(73) Assignee: Thennject. LLC, San Diego, CA (US)

(21) Appl. No.: 12/511,977

(22) Filed: Jul. 29, 2009

Related U.S. Application Data
(63) Continuation of application No. 12/411,655, filed on Mar. 26, 2009.
(60) Provisional application No. 61/123,815, filed on Apr. 10, 2008.

(51) Int. Cl. A61K 9/14; A61P 43/00

(52) U.S. Cl. 424/489

(57) ABSTRACT

Monocytes and dendritic cells are grafted to the surface of microparticles for use as a therapeutic device for stimulating a post-injection immune response in vivo. Various embodiments include an injectable composition, an apparatus for grafting cells to the surface of polymer microspheres, and methods of facilitating activation and delivery of an injectable therapeutic device are disclosed.

### CROSS-REFERENCE TO RELATED APPLICATIONS

This application claims benefit of priority of U.S. Provisional Application Ser. No. 61/123,815 titled “Dendritic Cell Therapy Using Pharmacologically Active Microcarriers” filed Apr. 10, 2008, the contents of which are hereby incorporated by reference. This application is a continuation of U.S. patent application Ser. No. 12/411,655 titled “SYSTEM AND COMPOSITION FOR DENDRITIC CELL THERAPY USING PHARMACOLOGICALLY ACTIVE MICROCARRIERS” filed Mar. 26, 2009, the contents of which are hereby incorporated by reference.

### FIELD OF THE INVENTION

This invention relates to the field of preparation and transplantation of biologically active substances for use as an immune response stimulant in a patient. More specifically, the invention relates to injectable microparticles fabricated from biocompatible materials and having surface-grafted monocytes and dendritic cells for differentiation and maturation in vivo upon transplantation into the tissue of a patient.

### BACKGROUND OF THE INVENTION

Monocytes are cells present in blood and various tissues which replenish resident macrophages and dendritic cells. The process by which monocytes differentiate into one of macrophage and dendritic cells is referred to as monocytic differentiation. Additionally, monocytes can move quickly (approx. 8-12 hours) to sites of infection in the tissues and differentiate into macrophages and dendritic cells to elicit an immune response.

Dendritic cells are involved in the initiation of both innate and adaptive immune responses. Dendritic cells are antigen-presenting cells (APC), which act as cellular sentinels in every tissue of the human body, by detecting foreign antigens that serve as molecular signals of pathogen invasion.

During the adaptive immune response, an immature dendritic cell engulfs an antigen (e.g., an antigen from a pathogen, tumor, infected cell or other abnormal cell, or a self-antigen), after which the dendritic cell undergoes a maturation process and migrates to a lymph node. Over the course of this maturation process, the foreign antigen is cleaved into small peptides within the dendritic cells. These peptides are bound to major histocompatibility complex (MHC) class I and II molecules and presented on the surface of the mature dendritic cells. By presenting such processed peptides to T cells and B cells within the lymph node, mature dendritic cells directly and indirectly activate various subsets of these and other cells of the immune system, thereby guiding a series of immune responses that ultimately lead to elimination of pathogens.

Microcarriers such as microspheres fabricated from various materials are widely known in the art. Microcarriers fabricated from biocompatible materials have been used for sustained release of therapeutics, bulking devices, and other medical applications. Extensive use of polymer microcarriers in the medical industry as well as extensive laboratory testing has provided a surplus of data relating to biocompatibility and toxicology.

One use for microparticles as described in US Patent Application Publication No. 2005/0129776 by Montero-Menei et al., is the preparation of polymer microparticles adapted to adhere cytokines and other adjuvants which can stimulate an immune response, the contents of which is hereby incorporated by reference. These microparticles can be adapted to adhere tumor antigens (such as those prepared from autologous tumor cells) and cytokines (such as GM-CSF, IL2, IL12 and IL18), which can be transplanted into a patient. Once in the body, these microparticles present the adhered antigens to native dendritic cells for subsequent maturation and migration to the patients' lymph nodes, wherein an immune response is generated. One problem with this method is time required to generate sufficient yield of autologous tumor cells from a tumor sample for adhering to microspheres. Autologous tumor cells are grown using various culture techniques and are substantially time consuming. Another problem is the reliance on native dendritic cells to successfully locate, bind, and present the targeted antigen (maturation), prior to successful migration to the lymph nodes.

Efforts to utilize dendritic cells for stimulation of an immune response for treatment of tumor pathogens among other invaders have been contemplated, however due the inherent lifespan of certain cells, the focus has been on attracting and differentiating native monocytic cells in vivo, see at least Warren et al., U.S. Patent Application Publication No. 2006/0078540, the contents of which is hereby incorporated by reference. Warren discloses a matrix for implantation which attracts and differentiates native monocytes, resulting in an influx of dendritic cells for the stimulation of an immune response.

It has been shown that certain polymer compositions, specifically PLGA poly (Lactic-co-Glycolic Acid) and other lactide-derived polymers, are able to release immunostimulant and/or antitumor cytokines (Golumbek et al., 1993; Mullerad et al., 2000; Pettit et al., 1997, Menei et al., 1996, 1999, 2000; Péan et al., 2000), their contents of which are hereby incorporated by reference. Additional examples of the relevance of certain polymers to the treatment of cancer can be found in U.S. Pat. No. 6,589,526 filed Jan. 31, 2006 titled “Enrichment of Dendritic Cells from Blood”, and U.S. Patent Application Publication No. 2001/0043914 filed Dec. 27, 2000 titled “Methods and Products for Tumor Immunotherapy”; the entire contents of which are hereby incorporated by reference.

Fabrication of PLGA and other microspheres is known in the art. Procedures based on ink-jet technology have been used to fabricate PLGA microspheres that incorporate chemical compounds as described in Fletcher et al., Talanta 76 (2008) 949-955, the contents of which are hereby incorporated by reference.

There remains a longstanding need for an improved method and composition for the treatment of tumors and pathogens which rapidly and efficiently induces an effective immunological response in vivo.

### SUMMARY OF THE INVENTION

The present invention provides a microsphere having a surface and an inner matrix, wherein one or more cells selected from the group consisting of monocytes and dendritic cells are grafted to the surface of the microsphere. Several microspheres each having a plurality of surface-grafted adherent cells are suspended in a solution for injection and injected into a targeted region, such as a tumor.

In one embodiment, the inner matrix of the microsphere comprises one or more biodegradable polymers. The inner matrix of the microsphere can be fabricated from a biocompatible polymer composition by any of the techniques known in the art, preferably by an emulsion evaporation or spray drying technique. The one or more biodegradable polymers can be capable of resorption by the tissue surrounding the implanted microsphere.

In another embodiment, the inner matrix comprises one or more agents for sustained release. The one or more agents for sustained release can be selected from the group consisting of cytokines (such as GM-CSF, IL-2, IL-4, IL-12, IL-1 8, IL-23), growth factors, TNF-Alpha, Nucleic Acids (such as DNA, RNA), and CD-40 Ligand, polypeptides, polysaccharides such as lipopolysacharides and endotoxins.

In another embodiment, the inner matrix of the microsphere comprises one or more hollow pores. These pores can be used to incorporate one or more agents for sustained release. The one or more agents for sustained release can be selected from the group consisting of cytokines (such as GM-CSF, IL-2, IL-4, IL-12, IL-18, IL-23), growth factors TNF-Alpha, Nucleic Acids (such as DNA, RNA), and CD-40 Ligand, polypeptides, polysaccharides such as lipopolysacharides and endotoxins.

In another embodiment, the inner matrix does not comprise agents for sustained release.

In another embodiment, the inner matrix of the microsphere comprises at least one of metallic nanoparticles or magnetic nanoparticles(such as paramagnetic or ferromagnetic nanoparticles). Metallic and or magnetic nanoparticles can render the inner matrix of the microsphere capable of magnetic separation from a solution in suspension. Once a magnetic field is applied, the above microspheres can be referred to as “magnetized microspheres”. Magnetized microspheres can be retained in an apparatus by way of a magnetizing force, thereby facilitating a magnetized centrifugation or flushing technique useful in separating microspheres from a solution.

In another embodiment, the microsphere can comprise a coating, such as a polymer coating. The coating can further comprise one or more agents for sustained release and can be fabricated from a biocompatible polymer, and can be biodegradable and bioresorbable.

In another embodiment, the invention relates to an apparatus for preparation and activation of microspheres having a surface and an inner matrix, wherein one or more adherent cells; selected from the group consisting of monocytes and dendritic cells, are grafted to the surface of the microsphere. The system comprises one or more captive walls enclosing a plurality of biocompatible microspheres, at least one port, and a means for separating the microspheres from a solution (i.e. filtration).

In one embodiment, at least one port can comprise a luer-lok, threaded or tapered fitting. The port can further engage one or more filters.

In another embodiment, the invention relates to a process for the preparation of microspheres having a surface and an inner matrix, wherein one or more cells selected from the group consisting of monocytes and dendritic cells are grafted to the surface of the microsphere.

In another embodiment, the invention relates to a process for the injection of microspheres having surface-grafted cells selected from the group consisting of monocytes and dendritic cells into a targeted injection site. The injected microspheres subsequently release dendritic cells and dendritic cell precursors such as monocytic cells to the targeted tissue for differentiation and maturation in vivo, thus provoking an immune response at the targeted injection site. In the preferred embodiment, injection or transplantation of monocytic cells would be made to sites where targeted antigens are present, such as a tumor.

### BRIEF DESCRIPTION OF THE DRAWINGS

Other advantages and characteristics of the invention will become apparent from the examples illustrated below pertaining to a composition for transplantation to a targeted delivery site in the general field of medicine in which reference will be made to the attached figures in which:

FIG. 1 is a perspective view of an activated injectable therapeutic device according to one embodiment of the invention.

FIG. 2 is a schematic illustrating one process for the manufacture of polymer microspheres in accordance with one of the embodiments of the invention.

FIG. 3 illustrates a polymer microsphere having a substantially smooth surface.

FIG. 4 illustrates a polymer microsphere having a substantially smooth surface and further comprising a plurality of bio-active agents.

FIG. 5 illustrates a polymer microsphere having a plurality of pores on the surface of the bead, for which bio-active agents can be attached.

FIG. 6 illustrates a polymer microsphere having a plurality of pores on the surface of the bead, and a plurality of bio-active agents dispersed therein.

FIG. 7 illustrates an apparatus for the captivation and activation of a therapeutic device in accordance with one embodiment of the invention.

FIG. 8 illustrates an apparatus for the captivation and activation of a therapeutic device in accordance with another embodiment of the invention.

FIG. 9 illustrates a syringe system including a syringe and an apparatus for the captivation and activation of a therapeutic device according to aspects of the invention.

FIG. 10 illustrates an actioned syringe system in accordance with one embodiment of the invention.

FIG. 11 illustrates an actioned syringe system in accordance with another embodiment of the invention.

FIG. 12 is a perspective drawing of an activated therapeutic device comprising a plurality of dendritic cells according to aspects of the invention.

FIG. 13 is a perspective drawing of an activated therapeutic device comprising a plurality of dendritic cells and monocytes according to aspects of the invention.

FIG. 14 illustrates a general method for the captivation and activation of a therapeutic device for transplantation into a targeted delivery site.

FIGS. 15(a-b) illustrate embodiments for the retention of microspheres during evacuation of residual blood.

FIG. 16 is a photograph of a microsphere having a plurality of surface-grafted adherent cells on the surface.

### DETAILED DESCRIPTION

In the following description, for purposes of explanation and not limitation, details and descriptions are set forth in order to provide a thorough understanding of the present invention. However, it will be apparent to those skilled in the art that the present invention may be practiced in other embodiments that depart from these details and descriptions.

The therapeutic device, system for preparation thereof and methods described in accordance with embodiments of the present invention provide a means for effectuating a treatment of tumors and pathogens by rapidly and efficiently inducing an effective immunological response in vivo.

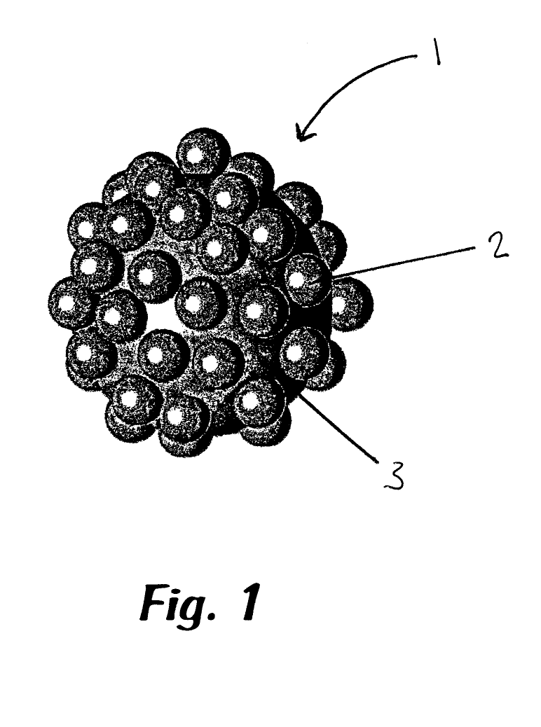

One aspect of this invention, as shown in FIG. 1, provides a microsphere 1 having a surface 3 and an inner matrix, wherein one or more cells 2 selected from the group consisting of monocytes and dendritic cells are grafted to the surface 3 of the microsphere. For purposes of this application, the term surface-grafted refers to cells which are attached, bound, connected or otherwise affixed to the surface of a polymer microsphere.

Because dendritic cells exist in various stages of differentiation and maturation, it is important to specify that immature dendritic cells are preferred for grafting to the microsphere. Immature dendritic cells are cells that have the capacity to differentiate into mature dendritic cells that can actively present antigen(s) on the cell surface. A plurality of such microspheres comprising at least one adherent cell grafted to the surface of the microsphere is then suspended in a suspension agent. A suspension agent can be any biocompatible solution for injection including water (Water for injection, WFI), saline solution, physiological buffer (phosphate buffered saline), or any other injectable biocompatible liquid, gel (such as collagen or gelatin) or sol. Upon delivery of the microsphere, a targeted region such as a tumor site becomes saturated with an increased concentration of monocytes and dendritic cells. As the cells leave the surface of the microsphere, usually via hydrolysis-degradation or polymer erosion, the cells encounter antigens from the local tumor region and begin the process of differentiation and maturation into antigen presenting cells (APC) which migrate to the lymph nodes to stimulate an immune response. The microsphere can further comprise one or more agents useful in the differentiation and maturation of these monocytes and dendritic cells.

There are two types of monocytes in peripheral blood: (i) the classical monocyte, which is characterized by high level expression of the CD14 cell surface receptor (CD14++ monocyte) and (ii) the non-classical, pro-inflammatory monocyte with low level expression of CD14 and with additional co-expression of the CD16 receptor (CD14+CD16+ monocyte). The CD14+CD16+ monocytes develop from the CD14++ monocytes, i.e. they are a more mature version. After stimulation with microbial products the CD14+CD16+ monocytes produce high amounts of pro-inflammatory cytokines like tumor necrosis factor and interleukin-12.

For purposes of this invention, the term adherent cell is offered to describe variations of peripheral blood monocytes and dendritic cells, these cells displaying a tendency to adhere to polymer surfaces and being capable of differentiation into antigen presenting cells and maturation in vivo.

The invention incorporates the use of biocompatible microspheres as a microcarrier for monocytes and dendritic cells which can be used to stimulate a post-injection immunological response in vivo.

### Preparation of Microspheres

Several methods for the fabrication of microspheres are known in the art. Among these include solvent evaporation (extraction), phase separation, and spray drying. Although microspheres can be fabricated by any one of these techniques, solvent evaporation (extraction) can be selected as a preferred method because controlled particle sizes in the nanometer to micrometer range can be achieved while yielding high encapsulation efficiencies and low residual solvent content.

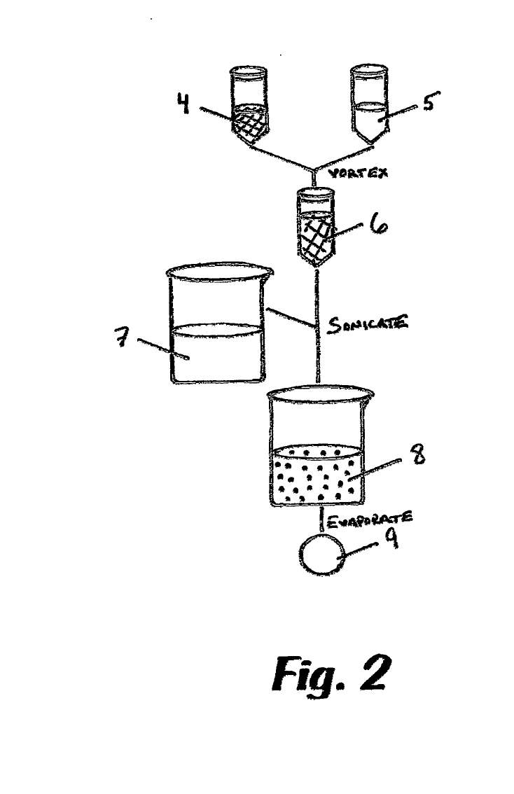

Microspheres can be prepared by a general solvent evaporation technique (extraction), as shown in FIG. 2, comprising the steps of: (i) dissolution or dispersion of one or more optional bio-reactive agents 4 in a solvent containing the polymer matrix material 5 to create a dispersed phase 6 and; (ii) emulsification of this dispersed phase 6 in an immiscible continuous phase 7; (iii) extraction of the solvent from the dispersed phase 6 by the continuous phase 7, which is optionally accompanied by solvent evaporation, either one transforming the droplets into solid microspheres 9; (iv) harvesting and drying of the microspheres 9 using a filter or other instrument.

Bio-reactive agents can be added to the solution of the matrix material by either codissolution in a common solvent, dispersion of finely pulverized solid material or emulsification of an aqueous solution of the bioactive compound immiscible with the matrix material solution, or other techniques known in the art. Examples of bio-reactive agents that aid in dendritic cell differentiation and maturation include: cytokines such as Granulocyte Monocyte Colony Stimulating Factor (GM-CSF), Interleukin-1 beta (IL-1β), Interleukin-2 (IL-2), Interleukin-4 (IL-4), Interleukin-6 (IL-6), Interleukin-12 (IL-12), Interleukin-18 (IL-18), Interleukin-23 (IL-23), Interferon-alpha (IFNα), Interferon-gamma (IFNγ), growth factors, Tumor Necrosis Factor-Alpha (TNF-A), Nucleic Acids (such as DNA, RNA), and CD40 Ligand, prostaglandin E₂ (PGE₂), polynucleotides, polypeptides, polysaccharides such as lipopolysacharides and endotoxins.

CD40 Ligand is a protein that is primarily expressed on activated T cells and is a member of the TNF family of molecules. It binds to CD40 on antigen-presenting cells (APC), which leads to many effects depending on the target cell type. In general, CD40L plays the role of a costimulatory molecule and induces activation in APC in association with T cell receptor stimulation by MHC molecules on the APC. The protein encoded by this gene is expressed on the surface of T cells. It regulates B cell function by engaging CD40 on the B cell surface. A defect in this gene results in an inability to undergo immunoglobulin class switch and is associated with hyper-IgM syndrome.

These agents are incorporated into the microsphere for the purpose of promoting the maturation of differentiated monocytes (i.e., dendritic cells) and to enhance the immune response generated by the dendritic cells. A sustained release of cytokines can provide the ideal environment for the maturation of transplanted and native dendritic cells. In this case, the desired outcome would be an anti-tumor response. For example, it has been shown that GM-CSF and GM-CSF+IL-4 will promote dendritic cell maturation in vitro.

In one embodiment, a matrix material solution is prepared by dissolving PLGA in an organic solvent. Various bio-reactive agents are added to the solution, preferably cytokines and growth factors. The solution is emulsified in water, and the solvent is evaporated. The remaining microspheres are then dried and separated according to size using one or more sieves. Those microparticles having a size between 10 μm and 300 μm and more preferably between 20 μm and 100 μm are selected for use. In this embodiment, the microspheres generally exhibit a substantially smooth surface.

In another embodiment, hollow porous microspheres can be fabricated by dissolving a reactive-polymer in a solvent, such as an organic solvent. A reactive-polymer is herein defined as a polymer composition having at least one leaving group on the backbone of the polymer structure (e.g. an alkoxy leaving group), where the leaving group being capable of forming a gas (e.g. CO₂). In this embodiment, the matrix material is similarly emulsified in water, and the solvent is evaporated. The leaving group can become stimulated to leave the polymer backbone upon contact with a catalyst such as heat. As the leaving group leaves to form a gas, tiny bubbles are created in the emulsified beads, and upon solvent evaporation/extraction, hollow porous microspheres are fabricated.

In one preferred embodiment, a matrix material solution is prepared by dissolving PLGA in an organic solvent such as dichloroethane (1,2 DCA, DCE). The solvent is then jetted through a nozzle sensitive to piezo electric distortion at frequencies and at pressures that break up the jetted stream into droplets. The uniformity of the droplets is controlled by the jetting parameters and catch solution constituents (e.g. water with a low percentage by weight (0.1-1% of polyvinyl alcohol). The jetting is preferably done while the nozzle is submersed into an aqueous reservoir such as a beaker or flask while the water is constantly stirred during the jetting process. The process can be accomplished using an inkjet printer system as described in recent publications (Fletcher et al., Talanta 76 (2008) 949-955). The water is stirred overnight (approximately 15 hours) to allow evaporation of the solvent and to harden the microspheres. The microspheres are then collected on a filter of average pore size that is less than the diameter of the microspheres or alternatively the microspheres are centrifuged to concentrate them in an appropriate vessel and the supernatant removed. The microspheres can be dried under vacuum to remove residual solvent. The microspheres are then hydrated and re-suspended in physiological buffer for dispensing into filter devices for veterinary or clinical use.

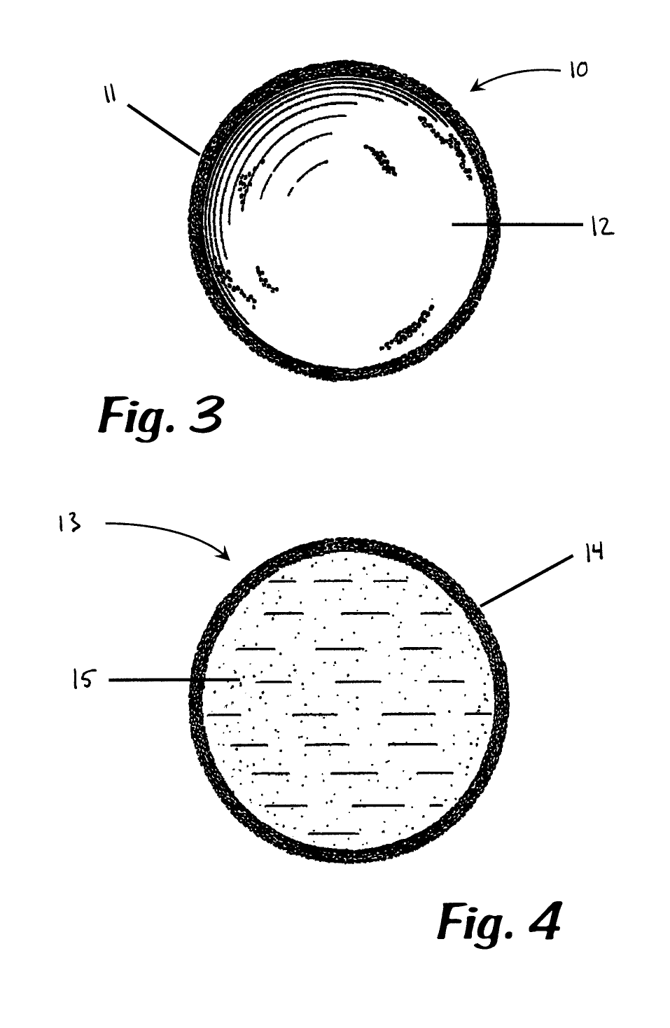

FIG. 3 illustrates a microsphere 10 having a surface 11 and an inner matrix 12. The surface 11 of the microsphere 10 is substantially smooth and substantially free from pores. The inner matrix 12 comprises a biocompatible polymer such as PLGA.

Optionally, as illustrated in FIG. 4, a microsphere 13 may comprise a surface 14 and an inner matrix 15, wherein one or more bio-active agents can be incorporated into the inner matrix 15 of the microsphere 13.

In the embodiment of FIG. 5, a hollow porous microsphere 16 comprises a surface 17, an inner matrix 18, and one or more pores 19. The one or more pores 19 are useful in adhering or otherwise incorporating additional agents into the microsphere such as bioactive agents.

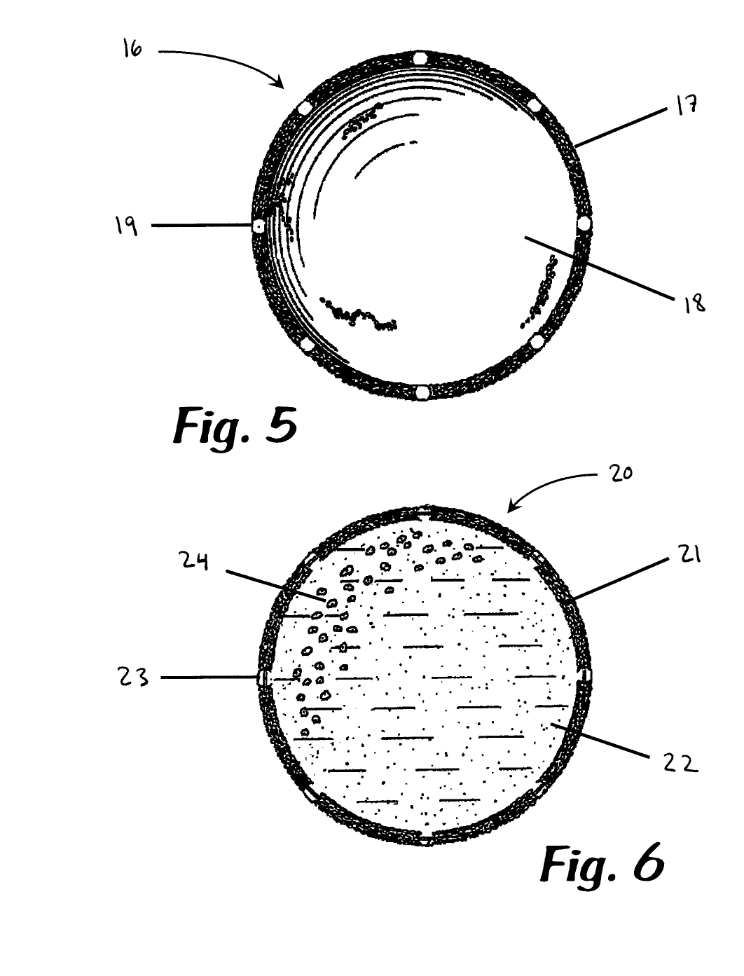

FIG. 6 illustrates a hollow porous microsphere 20 comprising a surface 21, an inner matrix 22, a plurality of pores 23, cytokines and other bio-active agents 24. The bioactive agents incorporated into the inner matrix 22 can be used to promote the development and maturation of dendritic cells. The sub-surface within each pore can further comprise one or more bioactive agents attached thereto. This can allow coating of porous microspheres with a solution containing a bioactive agent. The coating can bind to the pore site at the sub-surface, thereby providing a microsphere having an active agent at the sub-surface of the pores for sustained release.

### Grafting of Monocytes and Dendritic Cells to Surface of Microspheres

Human blood, in addition to red blood cells, contains peripheral blood mononuclear cells (PBMC) comprised of populations of adherent cells and non-adherent cells. Often these populations are separated by incubating the cells in tissue culture plates. The adherent cells settle and stick to the surface of the plate while the non-adherent cell population remains in suspension and can be removed by aspiration with a pipette along with the culture supernatant, leaving the adherent cell population on the surface of the plate. Similarly, adherent cells can attach themselves to microspheres and other solid particles in suspension. FIG. 16 is a photograph taken using a standard laboratory microscope in which a plurality of monocytic cells were successfully grafted to the surface of a polymer microsphere. The microsphere in this photo generally comprises an 85:15 composition of poly lactide:glycolide and fabricated by jetting into a 1% solution of polyvinyl alcohol (PVA) in water with 0.1% Gluteraldehyde. Other compositions of polymer have been tested and yield similar results.

Cellular adherence is herein defined as the degree to which peripheral blood adherent cells attach to a specific surface. It has been determined that cellular adherence is improved in the presence of chitosan, animal-derived collagen, and gelatin. Chitosan is a linear polysaccharide composed of randomly distributed β-(1-4)-linked D-glucosamine and N-acetyl-D-glucosamine. Collagen can be partially hydrolysed to yield gelatin, which has also been shown to increase cellular adherence.

In one embodiment, at least one of chitosan, collagen or gelatin is incorporated into the polymer microsphere by melting the additive slightly above its respective melting point (generally between 25° C.-45° C.) and blending with the polymer in a solvent suspension prior to solvent evaporation.

In another embodiment, a coating is applied to the polymer microsphere wherein the coating comprises at least one of chitosan, collagen, or gelatin. The coating can be a polymer coating or other coating composition which is capable of substantially coating the surface of the microsphere.

Monocyte-derived dendritic cells can be generated in vitro from peripheral blood mononuclear cells (PBMCs). Plating of PBMCs in a tissue culture flask permits adherence of monocytes. Treatment of these monocytes with interleukin-4 (IL-4) and granulocyte-macrophage colony stimulating factor (GM-CSF) leads to differentiation to immature dendritic cells (iDCs) in about a week. Subsequent treatment with tumor necrosis factor alpha (TNFa) can further differentiate the iDCs into mature dendritic cells.

In one aspect of the invention, a polymer microsphere is provided and a peripheral blood sample is introduced. The blood sample contains a number of adherent and non-adherent cells. The adherent cells can adhere to the surface of the polymer microsphere in the same manner as with a petri dish. Once adherent cells adhere to the surface of the microsphere (grafting), the residual blood is washed and the microspheres having one or more grafted adherent cells are suspended in a suspension agent. The suspension comprising microspheres and adherent cells is then incorporated into a syringe delivery system and injected into a targeted delivery site (i.e. a tumor). Once injected, the microsphere can degrade by hydrolysis or polymer bulk erosion. Adherent cells are released into the tumor region where they encounter tumor antigens. The adherent cells can differentiate and mature into antigen presenting cells (APCs) such as dendritic cells and migrate to the lymph nodes where an immune response is generated. Anti-tumor cells can then be produced by the host and naturally migrated to the tumor site.

In one embodiment, an apparatus for the captivation and activation of an injectable therapeutic device is provided. The apparatus comprises at least one captive wall member enclosing a plurality of biocompatible microspheres, at least one port, and a filter.

The captive wall member can be any geometric shape capable of captively surrounding or enclosing a plurality of microspheres. One having ordinary skill in the art and in combination with this disclosure will appreciate that a captive wall member can be fabricated from square or triangular tubing, a bag such as a blood bag, a syringe body, or other material.

In another embodiment, one captive wall member forms a cylindrical tube such as a syringe body. The syringe body can comprise a captive filter or external filter for the restriction of microspheres during evacuation of flowable contents. The syringe can further comprise one or more ports adjacent to the syringe body to function as an inlet or evacuation point for flowable contents.

In additional embodiments, three or more captive wall members can form an extruded geometric shape, such as square or triangle tubing. The tubing can further comprise a captive filter or an external filter. Additionally, the tubing can comprise one or more ports to function as an inlet or evacuation point for residual flowable contents.

A port provides the apparatus with a source for filling and evacuating fluid within the apparatus. The port is generally an extruded annular shape having one of a luer-lok, threading, or tapered fitting on a distal end. It is important that the port be adapted to engage with one of a syringe, needle, or tubing.

In one embodiment, the apparatus comprises two ports, each on opposite ends of the captive wall member and having a luer-lok fitting. A first port is adapted to engage with a luer-lok syringe and a second port is adapted to engage with one of a cannula or a filter having a conjugal Luer-lok fitting.

The filter can comprise a captive filter or an attached filter, generally having a pore size of less than 50 microns, more preferably 20 microns such as to allow the flow of residual fluid and certain flowable particulate while retaining microspheres within the apparatus.

In one embodiment, the filter is a captive filter, which for purposes of this invention is defined as a filtering means substantially enclosed within one or more captive walls of the apparatus for collection and activation of an injectable therapeutic device.

In another embodiment, the filter is an external filter, having a means to attach to at least one apparatus port. For example, an external filter might be a filter enclosed within a separate body having a luer-lok adapter for connecting to the apparatus.

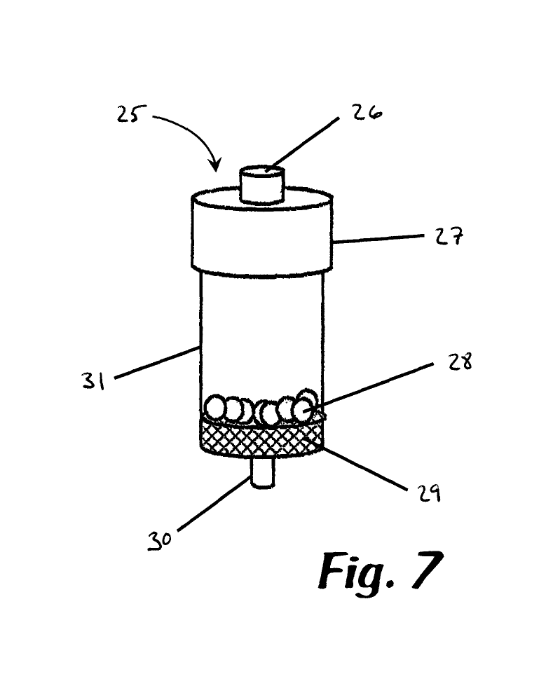

FIG. 7 illustrates an apparatus 25 for the captivation and activation of an injectable therapeutic device. In this preferred embodiment, the apparatus comprises a cylindrical captive wall member 31 containing a plurality of microspheres 28. The microspheres can be dried or alternatively suspended in water, saline or other biocompatible solution. The apparatus has a first port 30 and a second port 26. The first port 30 comprises a tapered fitting and the second port 26 comprises a luer-lok fitting. The cylindrical captive wall member 31 is sealed with a cap 27 which is attached to the cylindrical captive wall member 31 using a crimping device and a seal. The seal can be an o-ring, an adhesive, or a friction-fit, and functions to maintain an air-tight bond between the cap 27 and the captive wall member 31. The apparatus further comprises a captive filter 29, which is a filter enclosed within the captive wall member 31 functioning to restrict the flow out of the first port 30.

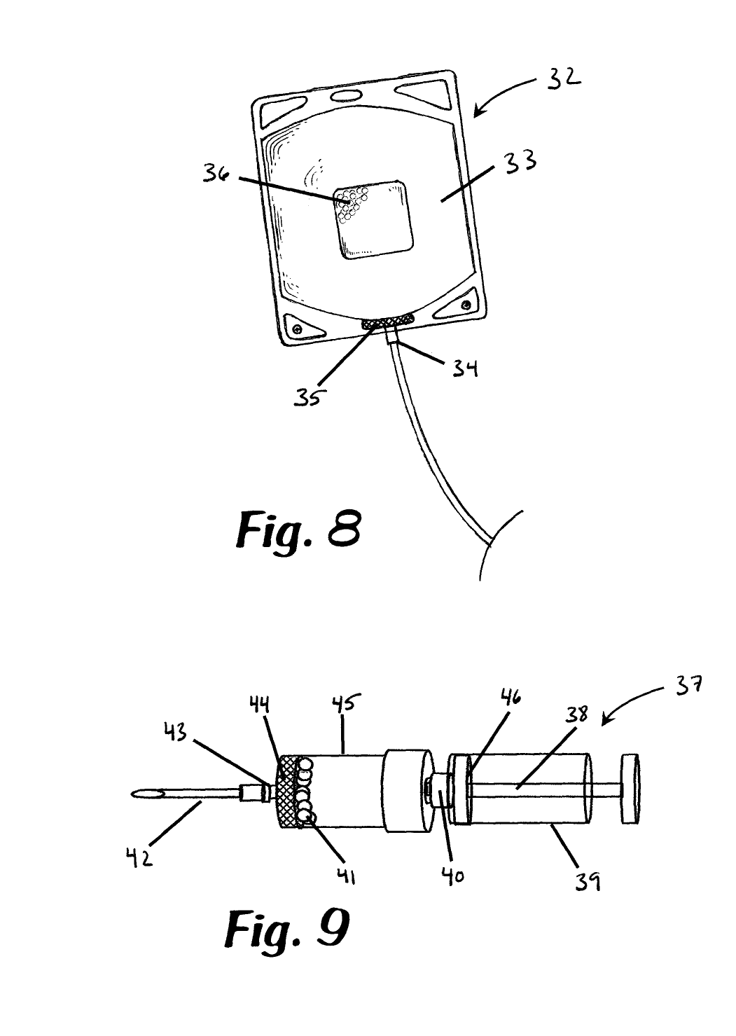

In an embodiment illustrated in FIG. 8, two captive wall members are thermo-sealed (impulse sealed) to form an apparatus for the captivation and activation of an injectable therapeutic device. The apparatus 32 comprises a captive wall member 33, a port 34, a captive filter 35, and a plurality of biocompatible microspheres 36. The captive wall member 33 captively encloses the microspheres 36. The captive filter 35 functions to retain the microspheres 36 within the apparatus 32 upon evacuation of flowable contents, i.e. residual blood. The port 34 functions as an inlet and evacuation point for flowable contents.

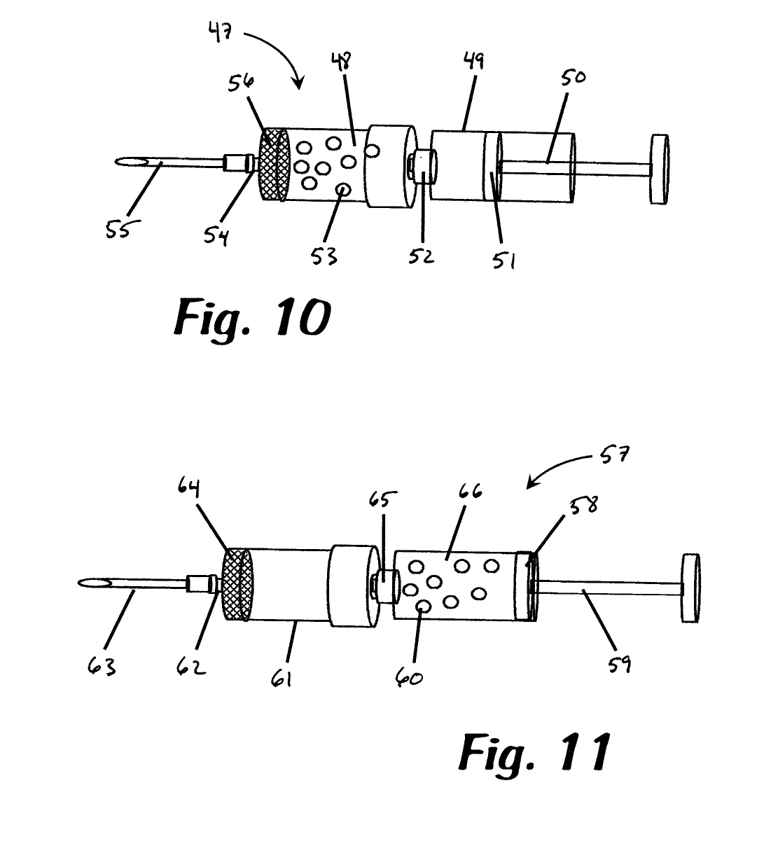

The apparatus described in FIG. 7 can further be combined with a syringe as shown in FIG. 9 to create a syringe system. FIG. 9 illustrates a syringe system 37 comprising; a syringe body 39, a plunger 46 and plunger rod 38, which is attached to an apparatus for the captivation and activation of an injectable therapeutic device at an engagement member 40. The engagement member 40 is the site where a syringe port is attached to an apparatus port by one of a luer-lok, tapered (friction) fitting, or threading. The apparatus further comprises; a cylindrical captive wall member 45, a captive filter 44, a distal port 43, and a plurality of biocompatible microspheres 41. The distal port 43 is attached to a needle. In this figure, the syringe is in a rest state, having drawn nothing into the apparatus. The syringe system can be used to harvest and graft adherent cells selected from the group consisting of, monocytes and dendritic cells, from the peripheral blood to the surface of the captively enclosed microspheres. After grafting adherent cells to the microspheres, they can be washed and transplanted into a targeted delivery site, such as a tumor. The grafted cells can then function to differentiate and mature to present antigens to the lymph nodes, thereby effectively inducing an immune response.

### Isolation and Delivery of Activated Microspheres

In a preferred embodiment, a filter is used to isolate activated microspheres (microspheres having surface-grafted adherent cells) in preparation for injection. Generally, the apparatus comprising a filter and microsphere beads is attached to a syringe, and the syringe is withdrawn so as to draw blood into but not through the apparatus. The syringe can then be removed and the apparatus capped to prevent loss of the sample through the port using a standard luer cap or threaded fitting. The apparatus is then placed on a roller, agitator, or other mechanical mixing device during exposure of the sample to the enclosed microspheres. Mechanical mixing prevents settling of the microspheres and mixes the sample to allow for greater exposure of cells to the surface of the enclosed microspheres. After a sufficient duration of time (usually about 1 hour), the microspheres will have adhered a number of monocytes and dendritic cells to their respective surface. A syringe; and where required-an external filter, is then connected to the apparatus. The sample is then evacuated or extruded from the apparatus through the filter. At this point it is preferred to rinse the enclosed microspheres using water or saline solution. A solution for injection can then be drawn into the apparatus thereby suspending the microspheres. The suspension comprising a solution for injection, and a plurality of microspheres having surface-grafted adherent cells can then be injected directly into the tissue at a desired site (e.g. tumor or surrounding tissue).

Optionally, a lysing agent can be introduced with the blood sample to ablate a targeted cell species (i.e. red blood cells) such that a higher yield of desired cells can be grafted on the surface of the microsphere carrier. For example, in an embodiment where a red blood cell lysing agent is introduced into a blood sample, a substantial quantity of red blood cells (RBC) will be ablated, leaving only white blood cells and other non-RBC cells in the population of which adherent cells remain. This can give a higher yield of desired adherent cells such as monocytes and dendritic cells.

FIG. 10 illustrates a syringe system 47 comprising an apparatus for the captivation and activation of an injectable therapeutic device, and a syringe. The syringe is attached to the apparatus using a luer-lok connection between conjugate ports 52 of the syringe and apparatus. A needle 55 is attached to the distal port 54 of the apparatus. The apparatus further comprises a captive filter 56 to prevent migration of the microspheres 53 out of the apparatus. The syringe plunger 51 and plunger rod 50 are actioned perpendicular to the syringe body 49, to create vacuum pressure and ultimately introduce peripheral blood 48 into the apparatus. Although some peripheral blood 48 may flow into the syringe, the syringe may be angled in a vertical direction to substantially maintain the microspheres 53 within the apparatus. The needle 55 and the syringe can then be removed and the apparatus capped. The apparatus can then be placed on a rocker during grafting of adherent cells to the surface of the microspheres.

In another embodiment as illustrated in FIG. 11, an apparatus containing a captive filter 64 and PLGA beads 60 is attached to a syringe to create a syringe system 57, and the syringe is withdrawn by pulling the plunger 58 and plunger rod 59, so as to draw a blood 66 through the apparatus 61 and into the syringe body. The syringe having taken up the beads can then be removed and capped to prevent loss of the sample using a standard luer cap or threaded fitting. Alternatively the syringe can remain attached to the apparatus 61 and the needle 63 removed from the distal port 62 of the apparatus 61, and capped.

The syringe is then placed on a roller, agitator, or other mechanical mixing device during exposure of the sample to the enclosed microspheres. Mechanical mixing prevents settling of the microspheres and mixes the sample to allow for greater exposure of cells to the surface of the enclosed microspheres. After a sufficient duration of time (usually about 1 hour), the microspheres will have adhered a number of monocytes and dendritic cells to their respective surface. The syringe is then connected to a filter apparatus (unless a captive filter containing apparatus remains attached to the syringe). The sample is then evacuated or extruded from the syringe through the filter apparatus. The filter retains the microspheres and the adherent cells that have attached to the microspheres. At this point it is preferred to rinse the enclosed microspheres using sterile physiological buffer (phosphate buffered saline) or saline solution (0.9% saline injection, usp). A solution for injection can then be drawn from the apparatus using a syringe thereby suspending the microspheres; the solution containing the adherent cells for injection can be injected directly into the tissue at a desired site (e.g. tumor or surrounding tissue).

In another embodiment, the microspheres can be isolated from the sample using a centrifugation device. Beads and cells of a particular buoyant density can be separated using gradients, step gradients, or separated based on a buoyant density that is different from red blood cells thus forming a layer either below or above the red blood cell layer (preferably below) after centrifugation in a clinical centrifuge or similar centrifuge for recovery from the red blood cells and non-adherent cells.

In another embodiment, the microspheres can be isolated from the sample using a magnetic or ferromagnetic separator. In this embodiment, metallic or magnetic nanoparticles can be incorporated into the matrix material during the preparation of microspheres, thus yielding microspheres having an inherent magnetic source. The syringe containing the metallic or otherwise magnetic particles is placed into a magnetic field which attracts the magnetized microspheres allowing their separation from the blood cells and non-adherent cells. The blood can be removed by pouring the blood from the syringe while the magnet is attached and the microspheres are immobilized.

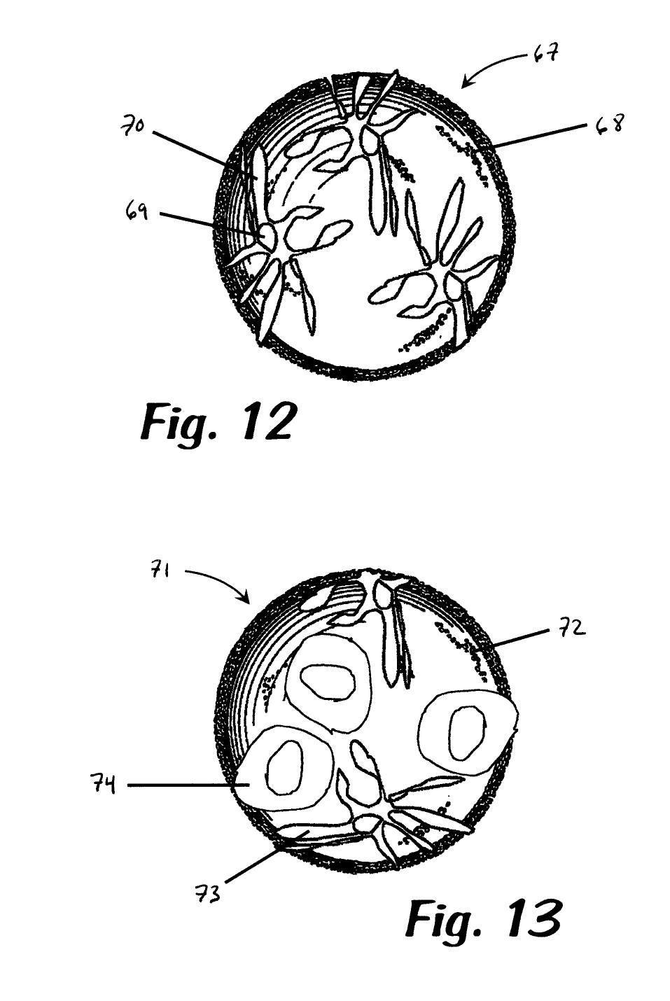

FIG. 12 illustrates an activated injectable therapeutic device 67 comprising; a biocompatible microsphere having a surface 68 and an inner matrix, where a plurality of dendritic cells are grafted to the surface of the microsphere. The dendritic cells have a nucleus 69, and dendrites 70. The inner matrix of the biocompatible microsphere can be fabricated using a PLGA or other biocompatible polymer.

FIG. 13 illustrates an activated injectable therapeutic device 71 comprising; a biocompatible microsphere having a surface 72 and an inner matrix, where a plurality of adherent cells selected from the group consisting of, monocytes 74 and dendritic cells 73, are grafted to the surface of the microsphere.

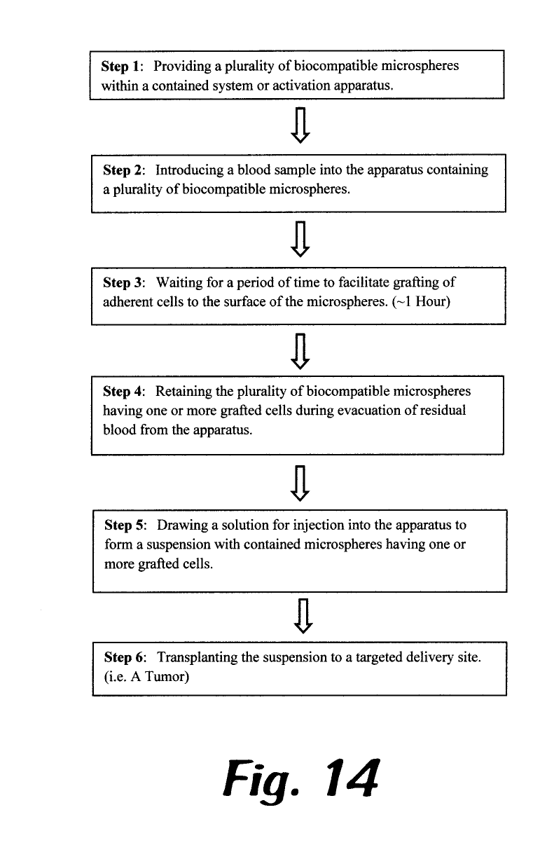

An activated injectable therapeutic device can generally be manufactured using the method illustrated in FIG. 14 and comprising the steps of; providing an apparatus containing a plurality of biocompatible microspheres, introducing a blood sample into the apparatus containing a plurality of biocompatible microspheres, waiting for a period of time to facilitate grafting of adherent cells to the surface of the microspheres, retaining the plurality of biocompatible microspheres having one or more grafted cells during evacuation of residual blood from the apparatus, drawing a solution for injection into the apparatus to form a suspension with contained microspheres having one or more surface-grafted cells, and delivering the suspension to a targeted delivery site, such as a tumor.

Certain techniques can be used to further separate adherent cells from non adherent cells prior to introduction of the blood sample to microspheres for grafting. By providing a high concentration of adherent cells in the blood sample being introduced, one can significantly improve grafting by (i) reducing the time require for binding, thereby improving the grafting technique, and (ii) enhancing the concentration of dendritic cell precursors by introducing a blood sample containing a high concentration of those cells.

One method for preparing the blood sample with an increased concentration of adherent cells in population can be accomplished by introducing a lysing agent to ablate undesired cells. For example a RBC lysing agent can be introduced to ablate RBC's within the blood sample. Another method is to use a centrifuge to separate cells according to a density gradient.

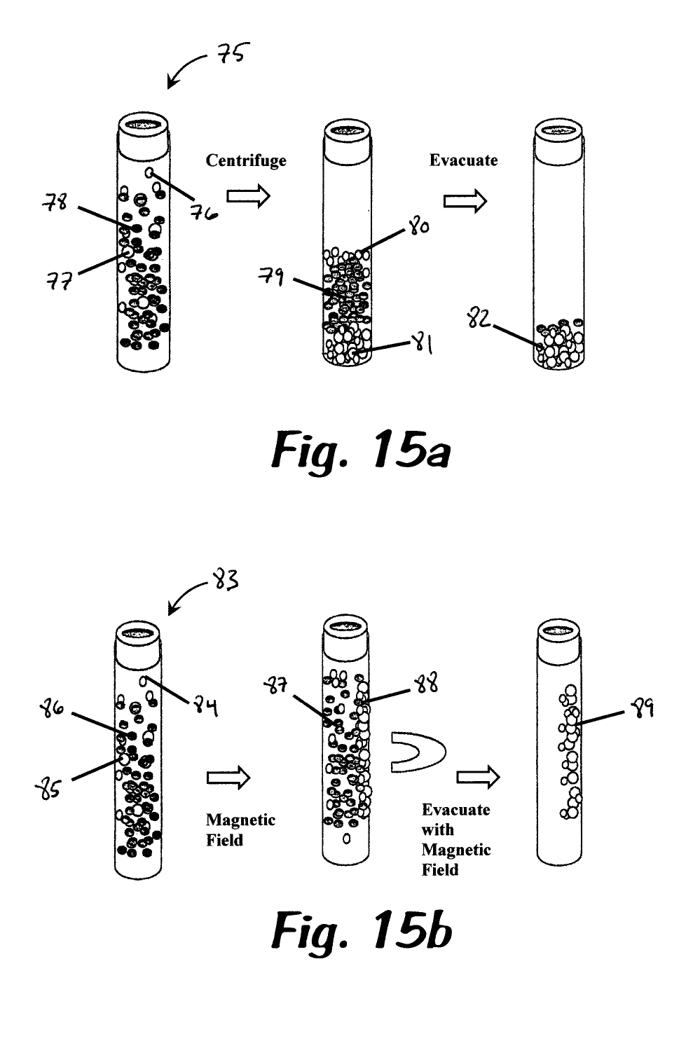

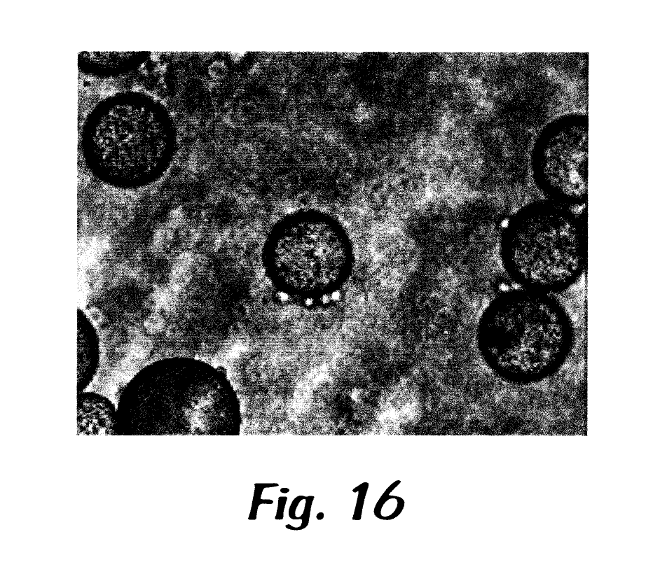

When evacuating residual blood and particulate, certain methods can be used to retain microspheres having surface-grafted cells. Of which, two methods are illustrated in FIGS. 15(a-b). FIG. 15a illustrates a blood collection tube 75 containing RBC's 78, white blood cells (WBC's) 76 and polymer microspheres 77. The blood collection tube 75 and contents are placed in a centrifuge to allow gradient separation. The gradient here comprises a layer of white blood cells 80, a layer of red blood cells 79, and a layer of microspheres having surface-grafted adherent cells 81. The top two layers can be evacuated with a pipette or other instrument, leaving a product substantially comprising polymer microspheres having surface-grafted adherent cells 82.

FIG. 15b illustrates a blood collection tube 83 containing RBC's 86, WBC's 84, and metallic or magnetic nanoparticle containing polymer microspheres having surface-grafted adherent cells 85. The blood collection tube 83 and contents are subjected to a magnetic field, whereby the microspheres and surface-grafted adherent cells are magnetically attracted and bound at the surface of the blood collection tube nearest the magnetic field 88. Residual blood cells 87 are not magnetically bound. While maintaining the magnetic field, residual blood is evacuated by decanting, pouring or other method. Subsequent to evacuation, a product substantially comprising polymer beads having surface-grafted adherent cells is isolated for further processing with a solution for injection.

### Methods for Transplantation of Adherent Cells

One unique aspect of the invention is the capture of adherent cells from the blood, grafting of the adherent cells to microspheres, suspending the microspheres and surface-grafted adherent cells into an injectable composition, and transplanting the microspheres having surface-grafted adherent cells to a targeted delivery site, such as a tumor. This method allows for rapid exposure of adherent cells to local antigens and rapid maturation of dendritic cells that ultimately leads to rapid and efficient stimulation of an immune response. Additionally, the suspension can be prepared and delivered in less than one hour, which drastically improves current techniques involving cell culture which can take several days.

In one embodiment, a method involves the steps of:
(i) providing a plurality of biocompatible microspheres each having a surface adapted to adhere one or more adherent cells, wherein said adherent cells are selected from the group consisting of monocytes and dendritic cells;
(ii) introducing a blood sample to said plurality of biocompatible microspheres;
(iii) allowing sufficient time for the grafting of one or more adherent cells from said blood sample to the surface of said biocompatible microspheres;
(iv) evacuating residual blood,
(v) suspending said microspheres in a solution for injection to form a suspension with said plurality of biocompatible microspheres having one or more grafted cells,
(vi) injecting said suspension to a targeted delivery site.

As previously described, adherent cells can be defined as dendritic cells and dendritic cell precursors such as monocytic cells or monocytes. Adherent cells, as their name would suggest, tend to naturally adhere to certain surfaces such as plastic or polymer surfaces (i.e. petri dishes and microspheres). There are thousands of polymer compositions which have the potential to adhere monocytes and dendritic cells; one having ordinary skill in the art can perform a simple test in about one hour to confirm whether a candidate polymer species is adapted to adhere these cells. In a simple version, one can introduce a peripheral blood sample to a particular polymer microsphere and allow about one hour for adhesion under ambient conditions, then filter the residual blood and observe the prospective polymer candidate under a microscope to confirm adhesion. As discussed above, certain additives can contribute to an improvement in cellular adhesion. Adherent cells can be found in peripheral blood and can be isolated from other peripheral blood cells using the aforementioned techniques, such as using a density gradient technique or by isolating the adherent cells on a different matrix or surface.

A sufficient time for the grafting of adherent cells to the surface of microspheres is that amount of time which is greater than 5 minutes and less than 15 hours. Although more time can be utilized, it would be disadvantageous as most of the cells will adhere before 15 hours of exposure. Preferably, a time of 1 hour will be sufficient to yield a useable concentration of adherent cells grafted to the surface of the microspheres.

In another embodiment, the above method can further include the step of introducing a cell lysing agent to ablate a species of blood cells. For example, red blood cells within the blood sample can be ablated for more efficient removal of RBCs and to prevent any interference with the binding of monocytes to the surface of the microsphere. This can be beneficial to produce a high yield of monocytes and dendritic cells grafted to the microsphere surface, and thus a more potent and efficient device for transplantation.

In another embodiment, the microspheres can comprise a metallic or magnetic (paramagnetic, ferromagnetic, or other magnetic) material. Subsequent to grafting of adherent cells, the microspheres can be retained by a magnetic source such as an electromagnet or stimulant of a magnetic field. While retaining the microspheres and grafted cells, residual blood can be evacuated, thus substantially retaining microspheres and grafted cells while flushing residual contents.

In another embodiment, a filter is used to retain the microspheres and grafted adherent cells. For best results in retaining the microspheres during filtration, a filter should have a maximum pore size of about three times the diameter of the microsphere. The optimal average pore size should be less than the dimensions of the microspheres.

In another embodiment, subsequent to evacuating residual blood, one or more washing steps can be incorporated in which the microspheres having adherent cells grafted the surface are washed and prepared for transplantation. The microspheres can be washed with water, saline solution, or any biocompatible rinsing solution. The microspheres and grafted adherent cells can then be suspended in a solution for injection, a biocompatible gel, sol or other carrier to form a composition. The composition can then be injected into a targeted delivery site using a syringe and needle.

The above examples are set forth for illustrative purposes and are not intended to limit the spirit and scope of the invention. One having ordinary skill in the art will recognize that deviations from the aforementioned examples can be created which substantially perform the same task and obtain similar results.

What is claimed is:

1. A method for effectuating the activation and delivery of a therapeutic device comprising;
providing a plurality of biocompatible microspheres each having a surface adapted to adhere one or more adherent cells, wherein said adherent cells are selected from the group consisting of peripheral blood monocytes and dendritic cells;
introducing a blood sample to said plurality of biocompatible microspheres, wherein said blood sample comprises at least one of peripheral blood monocytes and dendritic cells;
allowing sufficient time for the grafting of one or more adherent cells from said blood sample to the surface of said biocompatible microspheres;
evacuating residual blood,
suspending said microspheres in a solution for injection to form a suspension with said plurality of biocompatible microspheres having one or more grafted cells,
injecting said suspension to a targeted delivery site.

2. The method of claim 1, further comprising the step of introducing a cell lysing agent into said blood sample prior to introducing blood to said plurality of biocompatible microspheres.

3. The method of claim 1, wherein said blood sample comprises peripheral blood monocytes, and wherein said monocytes are isolated from red blood cells by one of: using a density gradient technique or by isolating the adherent cells on a different surface.

4. The method of claim 1, wherein said sufficient time is greater than 5 minutes and less than 15 hours.

5. The method of claim 1, further comprising the step of washing said biocompatible microspheres having one or more surface-grafted cells immediately after evacuating residual blood.

6. The method of claim 1, wherein said plurality of microspheres comprise a biocompatible and biodegradable polymer matrix.

7. The method of claim 6, wherein said plurality of microspheres further comprise at least one metallic or magnetic particle.

8. The method of claim 7, further comprising the step of introducing a magnetic field to retain said plurality of biocompatible microspheres prior to evacuating said blood sample.

9. The method of claim 1, wherein said targeted delivery site is a tumor.

10. The method of claim 1, wherein said residual blood is evacuated through a filter adapted to retain said plurality of biocompatible microspheres.
